(19) United States
(12) Patent Application Publication
(10) Pub. No.: US 2007/0135362 A1
(43) Pub. Date: Jun. 14, 2007

(54) Method for demethylating the 3'-dimethylamino group of erythromycin compounds

(76) Inventor: Yaoquan Liu, Castro Valley, CA (US)

Correspondence Address: KOSAN BIOSCIENCES, INC, 3832 BAY CENTER PLACE, HAYWARD, CA, 94588, US

(21) Appl. No.: 11/591,726

(22) Filed: Nov. 1, 2006

Related U.S. Application Data
(63) Continuation-in-part of application No. 11/416,519, filed on May 2, 2006.
(60) Provisional application No. 60/748,981, filed on Dec. 8, 2005.
(60) Provisional application No. 60/748,898, filed on Dec. 8, 2005.

(51) Int. Cl. C07H 17/08; A61K 31/7048

(52) U.S. Cl. 514/29; 536/7.1

(57) ABSTRACT

A method for demethylating the 3′-dimethylamino group of erythromycin compounds.

### CROSS-REFERENCE TO RELATED APPLICATIONS

This application (a) is a continuation-in-part of nonprovisional application Ser. No. 11/416,519, filed May 2, 2006, and (b) claims the benefit under 35 U.S.C. §119(e) of U.S. Provisional Applications Nos. 60/748,981, filed Dec. 8, 2005, and 60/748,898, filed Dec. 8, 2005, the disclosures of which are incorporated herein by reference.

### BACKGROUND OF THE INVENTION

1. Field of the Invention

This invention relates to a method for demethylating the 3′-dimethylamino group of erythromycin compounds.

2. Description of Related Art

Gastrointestinal (“GI”) motility regulates the orderly movement of ingested material through the gut to ensure adequate absorption of nutrients, electrolytes, and fluids. Proper transit of the GI contents through the esophagus, stomach, small intestine, and colon depends on regional control of intraluminal pressure and several sphincters, which regulate their forward movement and prevent back-flow. The normal GI motility pattern may be impaired by a variety of circumstances, including disease and surgery.

GI motility disorders include gastroparesis and gastroesophageal reflux disease (“GERD”). Gastroparesis, whose symptoms include stomach upset, heartburn, nausea, and vomiting, is the delayed emptying of stomach contents. GERD refers to the varied clinical manifestations of the reflux of stomach and duodenal contents into the esophagus. The most common symptoms are heartburn and dysphasia, with blood loss from esophageal erosion also known to occur. Other examples of GI disorders in which impaired GI motility is implicated include anorexia, gall bladder stasis, postoperative paralytic ileus, scleroderma, intestinal pseudoobstruction, irritable bowel syndrome, gastritis, emesis, and chronic constipation (colonic inertia).

Motilin is a 22-amino acid peptide hormone secreted by endocrine cells in the intestinal mucosa. Its binding to the motilin receptor in the GI tract stimulates GI motility. The administration of therapeutic agents that act as motilin agonists (“prokinetic agents”) has been proposed as a treatment for GI disorders.

The erythromycins are a family of macrolide antibiotics made by the fermentation of the Actinomycetes Saccharopolyspora erythraea. Erythromycin A, a commonly used antibiotic, is the most abundant and important member of the family.

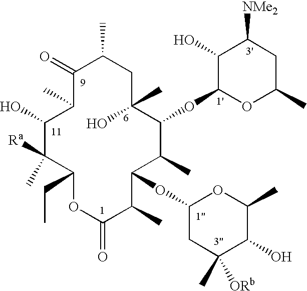

| | | | |
|---|---|---|---|
| (1) | Erythromycin A | R^{a} = OH | R^{b} = Me |
| (2) | Erythromycin B | R^{a} = H | R^{b} = Me |
| (3) | Erythromycin C | R^{a} = OH | R^{b} = H |
| (4) | Erythromycin D | R^{a} = H | R^{b} = H |

The side effects of erythromycin A include nausea, vomiting, and abdominal discomfort. These effects have been traced to motilin agonist activity in erythromycin A (1) and, more so, its initial acid-catalyzed degradation product (5). (The secondary degradation product, spiroketal (6), is inactive.)

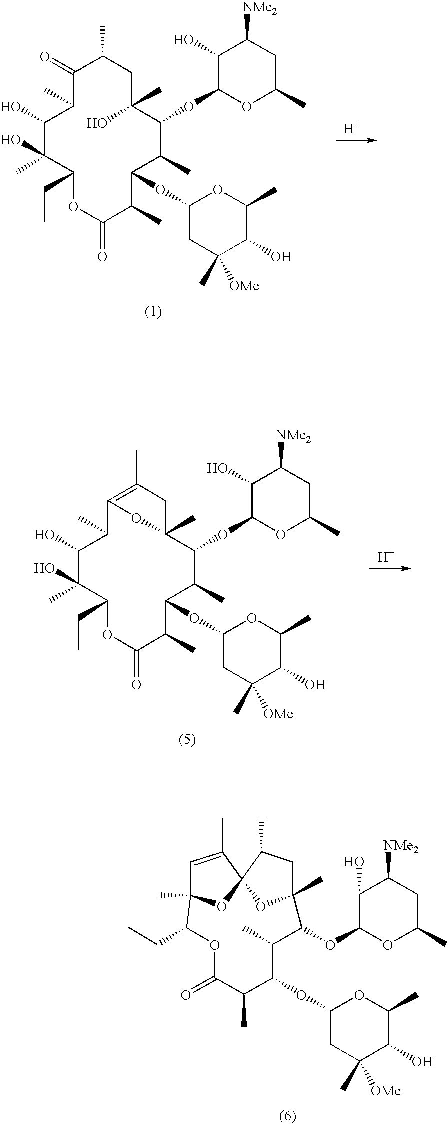

Spurred by the discovery of motilin agonist activity in erythromycin A and degradation product 5, researchers have endeavored to discover new motilides, as macrolides with prokinetic activity are called. Much of the research has centered on generating new erythromycin analogs, either via post-fermentation chemical transformation of a naturally produced erythromycin or via modification (including genetic engineering) of the fermentation process.

An important consideration in the development of new motilides is that they should have little or no antibiotic activity, lest they exert selective pressure on intestinal bacteria promoting the evolution of antibiotic-resistant strains. The 3′-dimethylamino group in the desosamine moiety of erythromycin is important for antibacterial activity. See Sakakibara and Omura, “Chemical Modification and Structure-Activity Relationship of Macrolides,” in Omura, ed., Macrolide Antibiotics: Chemistry, Biology, and Practice, pp. 85-89 (Academic Press 1984, Orlando, Fla.). Further, replacement of a methyl group with a larger ethyl or isopropyl group has been shown to result in compounds having prokinetic activity but little or no antibacterial activity—that is, a decoupling of the two types of activity. Tsuzuki et al., Chem. Pharm. Bull. 1989, 37 (10), 2687-2700. Quaternization of the dimethylamino group produces similar results. Sunazuka et al., Chem. Pharm. Bull., 1989, 37 (10), 2701-2709. These observations have led to modified 3′-dimethylamino groups as a recurring motif in motilides. See, for example: Omura et al., Omura et al, U.S. Pat. No. 5,008,249 (1991); Omura et al., U.S. Pat. No. 5,175,150 (1992); Harada et al., U.S. Pat. No. 5,470,961 (1995); Freiberg et al., U.S. Pat. No. 5,523,401 (1996); Freiberg et al., U.S. Pat. No. 5,523,418 (1996); Freiberg et al., U.S. Pat. No. 5,538,961 (1996); Freiberg et al., U.S. Pat. No. 5,554,605 (1996); Lartey et al., U.S. Pat. No. 5,578,579 (1996); Lartey et al., U.S. Pat. No. 5,654,411 (1997); Lartey et al., U.S. Pat. No. 5,712,253 (1998); Lartey et al., U.S. Pat. No. 5,834,438 (1998); Koga et al., U.S. Pat. No. 5,658,888 (1997); Miura et al., U.S. Pat. No. 5,959,088 (1998); Premchandran et al., U.S. Pat. No. 5,922,849 (1999); Keyes et al., U.S. Pat. N6,084,079 (2000); Ashley et al., U.S. Pat. No. 6,562,795 B2 (2003); Ashley et al., US2002/0094962 A1 (2002); Carreras et al., U.S. Pat. No. 6,875,576 B2 (2005); Ito et al., JP 60-218321 (1985) (corresponding Chemical Abstracts abstract no. 104:82047); Santi et al., U.S. Pat. No. 6,946,482 B2 (2005); Carreras et al., US2005/0119195 A1 (2005); Carreras et al., US2005/0119195 Al (2005); Liu et al., US2005/0256064 A1 ; Liu et al., U.S. Ser. No. 11/416,519, filed May 2, 2006; Gidda et al., U.S. Pat. No. 4,920,102 (1990); Omura et al., U.S. Pat. No. 4,948,782 (1990); Hoeltje et al., U.S. Pat. No. 5,418,224 (1995); Hoeltje et al., U.S. Pat. No. 5,912,235 (1999); Omura et al., U.S. Pat. No. 6,077,943 (2000); Ataka et al., U.S. Pat. No. 6,100,239 (2000); Jasserand et al.i, U.S. Pat. No. 6,165,985 (2000); Shimizu et al., WO 02/18403 (2002); Yoshida et al., WO 03/022289 A1 (2003); Omura et al., J. Antibiotics 1985, 38, 1631-2; Faghih et al., Biorg. & Med. Chem. Lett., 1998, 8, 805-810; Faghih et al., J. Med. Chem., 1998, 41, 3402-3408; Faghih et al., Drugs of the Future, 1998, 23 (8), 861-872; and Lartey et al., J. Med. Chem., 1995, 38, 1793-1798.

The modification of the 3 ′-dimethylamino group is accomplished by a two-step process. First, one of the methyl groups is removed (the demethylation step) and then the resulting monomethylamino group is alkylated with an alkylating agent RX (the alkylation step), where is a non-methyl alkyl group such as ethyl or isopropyl:

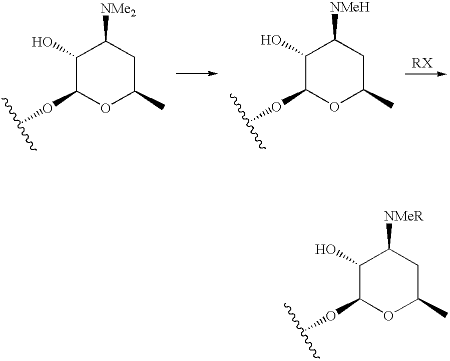

The conventional method for performing the demethylation step is to treat the dimethylamino compound with iodine in the presence of an oxy base such as alkali hydroxide, alkali methoxide, and the alkali salts of carboxylic acids such as sodium acetate, propionate, and benzoate. See Freiberg, U.S. Pat. No. 3,725,385 (1973) and Premchandran et al., U.S. Pat. No. 5,922,849 (1999). However, the prior art methods suffer from a number of limitations, as discussed hereinbelow.

The present invention provides an improved method for demethylating the 3′-dimethylamino group of erythromycin compounds.

### BRIEF SUMMARY OF THE INVENTION

In one aspect, this invention provides a method of preparing a compound II 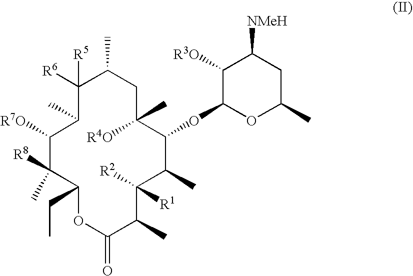
from a compound I 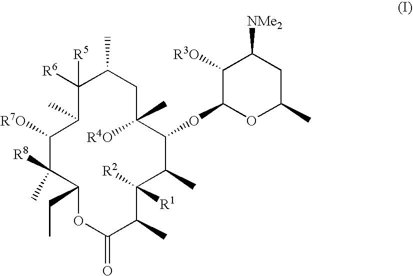
comprising treating compound I with iodine in the presence of an amine having a pK₅ in the range between about 5 and about 6;
wherein 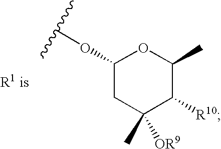
R² is H, or R¹ and R² combine to form ═O;
R³ is H or a hydroxyl protecting group;
R⁴ is H, C₁-C₄ alkyl, C₂-C₄ alkenyl, C₂—C₄ alkynyl, or a hydroxyl protecting group;
one of R⁵ and R⁶ is H and the other is OH, or R⁵ and R⁶ combine to form ═O or ═NOR¹¹;
R⁷ is H or a hydroxyl protecting group; and
R⁸ is H, OH, or protected hydroxyl;
R⁹ is H or a hydroxyl protecting group;
R¹⁰ is H, OH, or protected hydroxyl; and
R¹¹is H, C₁-C₄ alkenyl, C₂-C₄ alkenyl, or C₂-C₄ alkynyl.

### DETAILED DESCRIPTION OF THE INVENTION

Definitions

“Aliphatic” means a straight- or branched-chain, saturated or unsaturated, non-aromatic hydrocarbon moiety having the specified number of carbon atoms (e.g., as in “C₃ aliphatic,” “C₁-C₅ aliphatic,” or “C₁ to C₅ aliphatic,” the latter two phrases being synonymous for an aliphatic moiety having from 1 to 5 carbon atoms) or, where the number of carbon atoms is not specified, from 1 to 4 carbon atoms (2 to 4 carbons in the instance of unsaturated aliphatic moieties).

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like.

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E-(or Z-)2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

A protecting group, as in “protected hydroxyl” or “hydroxyl protecting group,” is a group that can be selectively attached to a hydroxyl group on a compound to render the hydroxyl group inert to certain chemical reaction conditions to which the compound is exposed and that, after such exposure, can be selectively removed. Many examples of hydroxyl protecting groups are known. See, for instance, Greene and Wuts, Protective Groups in Organic Synthesis, 3rd edition, pp. 17-245 (John Wiley & Sons, New York, 1999), the disclosure of which is incorporated herein by reference. Exemplary suitable hydroxyl protecting groups include for use with compounds of formula I include t-butyldimethylsilyl (“TBDMS” or “TBS”), triethylsilyl (“TES”) and triphenylsilyl (“TPS”).

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature), all stereoisomers are included within the scope of the invention, as pure compounds as well as mixtures thereof. Unless otherwise indicated, individual enantiomers, diastereomers, geometrical isomers, and combinations and mixtures thereof are all encompassed by the present invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

Compounds and Methods

Prior art methods for demethylation of the desosamine dimethylamino group suffer from several disadvantages. The demethylation reaction proceeds optimally at a pH range of around 8 to 9, but, as the reaction proceeds, hydrogen iodide is generated, tending to drive the pH of the reaction mixture downwards. But, if more basic reaction conditions are employed to compensate for the generation of hydrogen iodide, disproportionation of the iodine takes place. According to one prior art teaching, reaction pH is controlled by using sodium acetate and adding sodium hydroxide solution stepwise along with the iodine. According to the present invention, pH control is achieved more efficiently using an amine having a pK_{b} in the range of about 5 to about 6. We found that, in this way, the reaction was faster and only slightly more than a stoichiometric amount of iodine was needed.

Another potential complication addressed by the method of our invention is the fact that the demethylation reaction generates formaldehyde (Stenmark et al., J. Org. Chem., 2000, 65, 3875-3876), making the reaction a frequently incomplete equilibrium. Consequently, the reaction does not proceed to completion, leaving a residue of unreacted starting material that lowers yields and complicates product purification. Premchandran et al., U.S. Pat. No. 5,922,849 (1999) addressed this issue by either running the reaction in two stages or by sparging with an inert gas to remove the formaldehyde. In the method of our invention, the amine can react with the formaldehyde, thus driving the reaction towards completion.

Suitable amines for use in this invention are amines having a pK_{b} in the range of about 5 to about 6, at around ambient temperature (25° C.). Or, stated conversely, this means that the conjugate acid (protonated form) of the amine has a pKa in the range of about 8 to about 9, in view of the relationship
pKₐ=14−pK_{b}

In one preferred embodiment, the amine is a primary amine, a specific example of which is tris(hydroxymethyl)aminomethane (pK_{b} 5.9, also known as TRIS, THAM or tromethamine). In another preferred embodiment, the amine is a secondary amine, a specific example of which is morpholine (pK_{b} 5. 6).

Preferred compounds I that can be used in the method of this invention include erythromycin A (1), erythromycin B (2), clarithromycin (7, 6-O-methyl erythromycin A), and 9-dihydroerythromycin A (8, especially the 9-S stereoisomer).

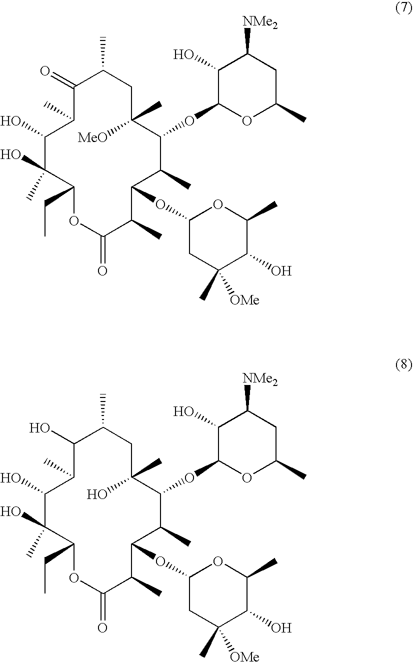

Suitable reaction solvents are methanol, aqueous methanol, dioxane, aqueous dioxane, THF, aqueous THF and the like, or mixtures thereof. Preferably, the solvent is methanol or aqueous methanol. The amount of amine can vary from 2 to 10 equivalents per equivalent of erythromycin derivative. The best results are obtained with about 5 equivalents of amine. Iodine (1.2-2 equivalents, preferably about 1.5 equivalents) is added in a single portion at the beginning. The reaction is generally carried out at temperatures from 40° C. to 70° C., and preferably from 50° C. to 60° C. The reaction is generally complete in 1-5 hours, depending on scale.

The method of this invention can be used to make N-demethyl erythromycin compounds, which, as noted above, can be further derivatized to make motilides having modified a 3′-dimethylamino group, such motilides being useful as prokinetic agents.

The practice of this invention can be further understood by reference to the following examples, which are provided by way of illustration and not of limitation.

General Procedures

Melting points were measured with Mel-Temp apparatus model 1001, thermometer uncorrected. Erythromycin A was purchased from NatroChem International. Tetrahydrofuran (THF) was distilled over sodium-benzophenone. All other reagents were purchased from Aldrich-Sigma and used without purification. ¹H NMR (400 MHz) and ¹³C NMR (100 MHz) spectra were recorded in CDC1₃ solution with a Bruker DRX 400 spectrometer. Chemical shifts were referred to δ 7.26 and 77.0 ppm for ¹H and ¹³C spectra, respectively. Thin layer chromatography plates was performed with Silica Gel 60 F plates, pre-treated with ammonia to neutralize any acidity of the silica gel. Flash chromatography was performed on Silica Gel 60. Both types of chromatographic media were from EMD.

### EXAMPLE 1

This example describes the preparation of (9S)-dihydroerythromycin A (9), a compound I that can be used in the method of this invention.

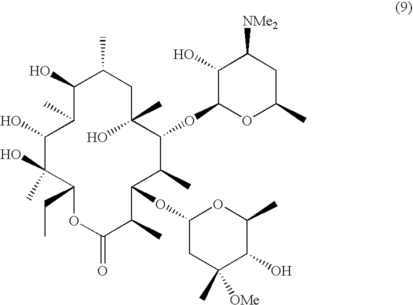

A 10-liter three-neck round bottom flask equipped with a mechanical stirrer and internal thermocouple probe was charged with methyl t-butyl ether (2,400 mL) and erythromycin A (400 g, 545 mmol, 1.0 eq. ). To this suspension was added MeOH (800 mL). The solution was stirred until became clear (ca. 5-15 min). The solution was cooled with an ice bath to an internal temperature of 2° C. Solid NaBH₄ (30.9 g, 816 mmol, 1.5 eq. ) was then added in one portion. The resulting suspension was stirred at 0° C. for 1 h, during which time the solution remained clear. After 1 h at 0° C. the ice bath was removed. The mixture was allowed to warm up to 22° C. and stirred for another 3 h. The mixture gradually became opaque. The reaction was complete as monitored by TLC (10% MeOH in CH₂Cl₂). Excess NaBH₄ was destroyed by careful addition of acetone (120 mL; exothermic reaction: acetone added at a rate to maintain an internal temperature of less than 30° C.) and phosphate buffer (5%, pH 6.0, 120 mL). The reaction turned to a clear solution with some white precipitate. Triethanolamine (400 mL) was added to help decompose the erythromycin-boron complex and the solution was stirred for 1 h. After adding saturated NaHCO₃ solution (3,200 mL), the mixture was extracted with EtOAc (3×2,000 mL). The combined extracts were washed once with water and once with brine (2,000 mL each), dried over solid Na₂SO₄. After removal of solvent, the crude product was dried in a vacuum oven (16 h, 50° C.). A white solid was obtained (416 g, mp 182-185° C.), which was used in the next step without further purification.

A small sample of compound (9) was purified by silica gel chromatography (1:1 acetone-hexane, 1% triethylamine). m/z: 737.0 (MH); ¹³C-NMR (CDCl₃):177.1, 103.3, 96.4, 84.4, 83.2, 79.3, 77.8, 77.7, 75.1, 74.5, 72.7, 70.8, 70.7, 69.4, 66.2, 65.1, 49.4, 45.6, 41.8, 40.4(2×), 37.0, 34.9, 34.3, 32.0, 28.9, 25.2, 21.7, 21.5, 21.2, 20.1, 18.1, 16.5, 15.1, 14.8, 11.2, 9.4 ppm.

### EXAMPLE 2

This example describes the demethylation of (9S)-dihydroerythromycin A (9) to produce N-desmethyl-(9S)-dihydro-erythromycin A (10).

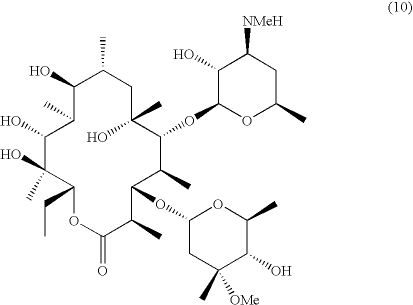

A six-liter three-neck round bottom flask equipped with a mechanical stirrer and internal thermocouple probe was charged with MeOH (2,000 mL), compound 9 from the previous example (150 g, theoretically 197 mmol, 1.0 eq. ) and tris(hydroxymethyl) amino-methane (119 g, 5 eq. ). The mixture was warmed to 55° C. internal temperature, during which all the materials dissolved. Iodine (75 g, 1.5 eq. ) was carefully added, at a rate to prevent the is slightly exothermic reaction from raising the internal temperature above 60° C. The mixture was stirred at 55° C. for 5 h. TLC (15% MeOH in CH₂Cl₂) indicated completion of the reaction. The reaction mixture was cooled to room temperature. Saturated sodium thiosulfate was used to destroy any excess iodine until the iodine color all disappeared. The mixture was concentrated by removal of about half of the MeOH, taking care to not remove too much of it - this causes precipitation of the product when aqueous solution is subsequently added, the precipitate being difficult to dissolve in the following extractions. The concentrate was diluted with aqueous NaHCO₃ (1,500 mL) and extracted with CH₂Cl₂ (3 ×1000 mL). The combined organic layers were washed once with water (1,500 mL) before drying over Na₂SO₄. The crude product 10 (113 g, mp 118-123° C.) was obtained after removal of solvent and drying in a vacuum oven (16 h, 50° C.). This material was suitable for use in subsequent synthetic procedures without further purification.

A pure sample of compound (10) was obtained after silica gel chromatography (2% to 10% methanol in dichloromethane, 1% triethylamine). m/z: 723.0 (MH); ¹³C-NMR (CDCl₃):176.4, 104.2, 97.6, 88.4, 82.6, 80.0, 78.3, 77.4, 74.6, 73.7(2×), 72.7, 71.6, 69.3, 66.5, 60.0, 49.3, 47.7, 41.7, 37.9, 36.5, 35.0, 34.0, 33.0, 32.1, 24.5, 21.9, 21.4, 20.8(2×), 17.9, 16.8, 16.0, 15.4, 11.1, 10.0 ppm.

### EXAMPLE 3

This example describes another synthesis of compound (10), using a different amine.

A solution of compound 9 (5.00 g, theoretically 6.79 mmol, 1.0 eq. ) and morpholine (2.96 mL, 5 eq.) in MeOH (70 mL) was warmed to 55° C. internal temperature. Iodine (2.59 g, 1.5 eq.) was carefully added in one portion. The mixture was stirred at 55° C. for 3 h. TLC (15% MeOH in CH₂Cl₂) indicated completion of the reaction. The reaction was worked up the same way as above to give the crude product 10 (3.95 g).

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

What is claimed is:

1. A method of preparing a compound II 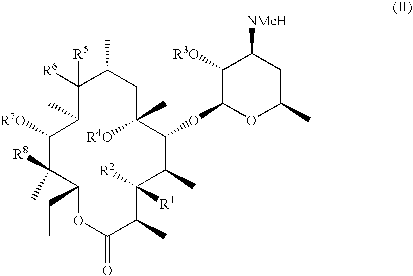
from a compound I 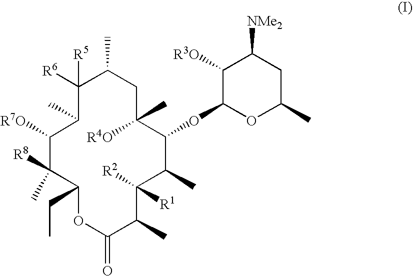
comprising treating compound I with iodine in the presence of an amine having a pK_{b} in the range between about 5 and about 6;
wherein 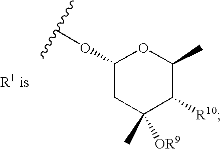
R² is H, or R¹ and R² combine to form ═O;
R³ is H or a hydroxyl protecting group;
R⁴ is H, C₁-C₄ alkyl, C₂-C₄ alkenyl, C₂-C₄ alkynyl, or a hydroxyl protecting group;
one of R⁵ and R⁶ is H and the other is OH, or R⁵ and R⁶ combine to form ═O or ═NOR¹¹;
R⁷ is H or a hydroxyl protecting group; and
R⁸ is H, OH, or protected hydroxyl;
R⁹ is H or a hydroxyl protecting group;
R¹⁰ is H, OH, or protected hydroxyl; and
R¹¹ is H, C₁-C₄ alkyl, C₂-C₄ alkenyl, or C₂-C₄ alkynyl.

2. A method according to claim 1, wherein compound I is erythromycin A, erythromycin B, clarithromycin, or 9-dihydroerythromycin A.

3. A method according to claim 1, wherein the amine is a primary amine.

4. A method according to claim 1, wherein the amine is tris(hydroxymethyl)-aminomethane.

5. A method according to claim 1, wherein the amine is a secondary amine.

6. A method according to claim 1, wherein the amine is morpholine.
